SEQUENCE LISTING (4 sequences)

SEQ ID NO: 1 - DNA, 1518 bp, human
  CDS (1)..(1518)
atgaagaaggccgaaatgggacgattcagtatttccccggatgaagacagcagcagctacagttccaacagcgacttcaactactcctaccccaccaagcaagctgctctgaaaagccattatgcagatgtagatcctgaaaaccagaactttttacttgaatcgaatttggggaagaagaagtatgaaacagaatttcatccaggtactacttcctttggaatgtcagtatttaatctgagcaatgcgattgtgggcagtggaatccttgggctttcttatgccatggctaatactggaattgctctttttataattctcttgacatttgtgtcaatattttccctgtattctgttcatctccttttgaagactgccaatgaaggagggtctttattatatgaacaattgggatataaggcatttggattagttggaaagcttgcagcatctggatcaattacaatgcagaacattggagctatgtcaagctacctcttcatagtgaaatatgagttgcctttggtgatccaggcattaacgaacattgaagataaaactggattgtggtatctgaacgggaactatttggttctgttggtgtcattggtggtcattcttcctttgtcgctgtttagaaatttaggatatttgggatataccagtggcctttccttgttgtgtatggtgttctttctgattgtggtcatttgcaagaaatttcaggttccgtgtcctgtggaagctgctttgataattaacgaaacaataaacaccaccttaacacagccaacagctcttgtacctgctttgtcacataacgtgactgaaaatgactcttgcagacctcactattttattttcaactcacagactgtctatgctgtgccaattctgatcttttcatttgtctgtcatcctgctgttcttcccatctatgaagaactgaaagaccgcagccgtagaagaatgatgaatgtgtccaagatttcattttttgctatgtttctcatgtatctgcttgccgccctctttggatacctaacattttacgaacatgttgagtcagaattgcttcatacctactcttctatcttgggaactgatattcttcttctcattgtccgtctggctgtgttaatggctgtgaccctgacagtaccagtagttattttcccaatccggagttctgtaactcacttgttgtgtgcatcaaaagatttcagttggtggcgtcatagtctcattacagtgtctatcttggcatttaccaatttacttgtcatctttgtcccaactattagggatatctttggttttattggtgcatctgcagcttctatgttgatttttattcttccttctgccttctatatcaagttggtgaagaaagaacctatgaaatctgtacaaaagattggggctttgttcttcctgttaagtggtgtactggtgatgaccggaagcatggccttgattgttttggattgggtacacaatgcacctggaggtggccat

SEQ ID NO: 2 - protein, 506 aa, human
MKKAEMGRFSISPDEDSSSYSSNSDFNYSYPTKQAALKSHYADVDPENQNFLLESNLGKKKYETEFHPGTTSFGMSVFNLSNAIVGSGILGLSYAMANTGIALFIILLTFVSIFSLYSVHLLLKTANEGGSLLYEQLGYKAFGLVGKLAASGSITMQNIGAMSSYLFIVKYELPLVIQALTNIEDKTGLWYLNGNYLVLLVSLVVILPLSLFRNLGYLGYTSGLSLLCMVFFLIVVICKKFQVPCPVEAALIINETINTTLTQPTALVPALSHNVTENDSCRPHYFIFNSQTVYAVPILIFSFVCHPAVLPIYEELKDRSRRRMMNVSKISFFAMFLMYLLAALFGYLTFYEHVESELLHTYSSILGTDILLLIVRLAVLMAVTLTVPVVIFPIRSSVTHLLCASKDFSWWRHSLITVSILAFTNLLVIFVPTIRDIFGFIGASAASMLIFILPSAFYIKLVKKEPMKSVQKIGALFFLLSGVLVMTGSMALIVLDWVHNAPGGGH

SEQ ID NO: 3 - DNA, 25 bp, Artificial Sequence
  Description of Artificial Sequence primer
ccgcggtcatagctgtttcctgaac

SEQ ID NO: 4 - DNA, 43 bp, Artificial Sequence
  Description of Artificial Sequence primer
cagctatgaccgcggttaatggccacctccaggtgcattgtgt

(19) United States
(12) Patent Application Publication
(10) Pub. No.: US 2021/0301262 A1
(43) Pub. Date: Sep. 30, 2021

(54) ANIMAL CELL, METHOD FOR PRODUCING ANIMAL CELL, AND METHOD FOR PRODUCING TARGET PROTEIN

(71) Applicant: FUJIFILM Corporation, Tokyo (JP)

(72) Inventors: Yuya WATANABE, Ashigarakami-gun (JP); Tatsuya MATSUURA, Ashigarakami-gun (JP)

(73) Assignee: FUJIFILM Corporation, Tokyo (JP)

(21) Appl. No.: 17/344,590

(22) Filed: Jun. 10, 2021

Related U.S. Application Data
(63) Continuation of application No. PCT/JP2019/048216, filed on Dec. 10, 2019.

(30) Foreign Application Priority Data
Dec. 11, 2018 (JP) 2018-231592

(51) Int. Cl. C12N 5/10; C07K 14/705; C12N 15/89

(52) U.S. Cl. CPC C12N 5/10; C12N 2510/02; C12N 15/89; C07K 14/705

(57) ABSTRACT

Provided are an animal cell capable of producing a target protein with high productivity, a method for producing the animal cell, and a method for producing a target protein using the animal cell. According to an aspect of the present invention, there are provided an animal cell that has a gene encoding a target protein and a foreign gene encoding SNAT2 and linked to a promoter and overexpresses SNAT2; a method for producing the animal cell; and a method for producing a target protein using the animal cell.

### CROSS-REFERENCE TO RELATED APPLICATIONS

This application is a Continuation of PCT International Application No. PCT/JP2019/048216 filed on Dec. 10, 2019, which claims priority under 35 U.S.C § 119(a) to Japanese Patent Application No. 2018-231592 filed on Dec. 11, 2018. Each of the above application(s) is hereby expressly incorporated by reference, in its entirety, into the present application.

### BACKGROUND OF THE INVENTION

### 1. Field of the Invention

The present invention relates to animal cells that express a target protein. An aspect of the present invention relates to a method for producing the above-mentioned animal cells, and a method for producing a target protein using the above-mentioned animal cells.

### 2. Description of the Related Art

In producing biopharmaceuticals such as antibodies, fed-batch culture, which improves a state of cells by adding nutrients to a culture solution, is often used to improve the productivity of antibodies. In addition, in producing biopharmaceuticals such as antibodies, a perfusion culture method, in which a culture solution is continuously filtered and discharged while a fresh medium containing nutrients is continuously fed to a culture tank, is often used to improve the productivity of antibodies.

WO2009/020144 discloses a method for producing polypeptide, including strongly expressing alanine aminotransferase, culturing cells in which DNA encoding a desired polypeptide is introduced, and producing the desired polypeptide, as a method capable of producing protein in a high production amount.

### SUMMARY OF THE INVENTION

In fed-batch culture, it is possible to perform cell culture for a long period and at a higher cell density compared to a case where nutrients are not additionally added, but there is a problem in that cell viability is degraded in the latter half of the culture due to accumulation of waste products secreted from cells, and thus products cannot be increased by extended culture period.

In addition, there is a problem in that in order to culture cells at high density in the perfusion culture method, the cost of the culture is increased since harvesting and supplying of a medium in an amount of 1 to 3 times the culture volume per day is required to supply nutrients and discharge waste products out of the system.

An object of an aspect of the present invention is to provide an animal cell capable of producing a target protein with high productivity. Another object of an aspect of the present invention is to provide a method for producing the animal cell and a method for producing a target protein using the animal cell.

The present inventors have conducted intensive studies to achieve the above-mentioned objects, and as a result, the present inventors found that the production amount of protein is affected by forcibly expressing a sodium-dependent neutral amino acid transporter-2 (SNAT2) gene (gene name is SLC38A2) in CHO cells, and thus it is possible to achieve improvement of antibody productivity (Qp) and improvement of antibody production amount. Aspects of the present invention was completed based on the above findings.

That is, according to an aspect of the present invention, the following inventions are provided.

<1> An animal cell that has a gene encoding a target protein and a foreign gene encoding SNAT2 and linked to a promoter, and overexpresses the SNAT2.

<2> The animal cell according to <1>, in which the overexpression is constant.

<3> The animal cell according to <1> or <2>, in which the foreign gene encoding SNAT2 has a base sequence having 90% or more sequence identity with a base sequence of SEQ ID NO: 1.

<4> The animal cell according to any one of <1> to <3>, in which the foreign gene encoding SNAT2 includes a base sequence of SEQ ID NO: 1.

<5> The animal cell according to any one of <1> to <4>, in which the animal cell is a CHO cell.

<6> A method for producing the animal cell according to any one of <1> to <5>, the method including a step of introducing a gene encoding a target protein and a foreign gene encoding SNAT2 and linked to a promoter, into an animal cell.

<7> The method according to <6>, in which the step of introducing a foreign gene encoding SNAT2 and linked to a promoter is performed by electroporation.

<8> A method for producing a target protein, including culturing the animal cell according to any one of <1> to <5>.

<9> The method according to <8>, in which the culture is fed-batch culture.

<10> The method according to <9>, in which a seeded cell density of a cell culture is 0.2×10⁶ cells/mL to 5×10⁶ cells/mL.

<11> The method according to <10>, in which a viable cell rate during a culture period is 60% or more over the entire period.

<12> The method according to <8>, in which the culture is perfusion culture.<

13> The method according to <12>, in which the seeded cell density of the cell culture is 0.2×10⁶ cells/mL to 1×10⁷ cells/mL.

<14> The method according to <13>, in which a viable cell rate during a culture period is 90% or more over the entire period.

According to the animal cell of an aspect of the present invention, it is possible to produce a target protein with high productivity.

### BRIEF DESCRIPTION OF THE DRAWINGS

FIG. 1 illustrates a result of measuring an antibody concentration in a culture supernatant of cells after gene introduction.

FIG. 2 illustrates a result of measuring antibody productivity (Qp) per cell in the culture supernatant of cells after gene introduction.

FIG. 3 illustrates a result of measuring a cell size after gene introduction.

FIG. 4 illustrates a result of measuring the cumulative number of viable cells during a culture period.

### DESCRIPTION OF THE PREFERRED EMBODIMENTS

Hereinafter, embodiments for carrying out an aspect of the present invention will be described in detail.

In the present specification, a numerical value range indicated by using “to” means a range including numerical values described before and after the “to” as a minimum value and a maximum value, respectively.

[Animal Cells]

An animal cell of an aspect of the present invention is an animal cell that includes a gene encoding a target protein and a foreign gene encoding SNAT2 and linked to a promoter, and overexpresses the SNAT2.

The expression of the SNAT2 gene activates the mTORC1/S6K pathway in a mouse liver (Nature Communications 6, Article number: 7940 (2015)), and this thus suggests a relationship between the expression of the SNAT2 gene and the expression of the mTOR gene. It has been reported that in rat cells, cell proliferation is suppressed by activation of mTOR and cell size is increased (Genes Dev. 2002 Jun 15; 16 (12): 1472-1487). In addition, it has also been shown that the larger the cell size, the greater the production amount of a protein product (Cytotechnology 34: 59-70, 2000). However, the relationship between SNAT2 gene expression and the production amount of recombinant protein is unclear.

In WO2009/020144, a decrease in survival rate was moderated due to the effect of introducing alanine aminotransferase, but since the period that survival rate of cells is 60% or less is long, there is concern about the influence on antibody quality. In addition, since the antibody productivity (Qp) per cell is lowered by the introduction of the taurine transporter, there is a problem that even if the cell density is increased, it does not lead to the production amount. On the other hand, in an aspect of the present invention, antibody productivity (Qp) per cell is improved by enhancing the expression of the SNAT2 gene involved in protein biosynthesis.

<Target Protein>

In an aspect of the present invention, the kind of the target protein is not particularly limited, and examples thereof include a recombinant polypeptide chain, a recombinant secreted polypeptide chain, an antigen-binding protein, a human antibody, a humanized antibody, a chimeric antibody, a mouse antibody, a bispecific antibody, and an Fc fusion protein, a fragmented immunoglobulin, and a single-chain antibody (scFv). The target protein is preferably a human antibody, a humanized antibody, a chimeric antibody, or a mouse antibody. Examples of the fragmented immunoglobulin include Fab, F(ab′)₂, Fv, and the like. The class of the antibody is also not particularly limited, and may be any one class of IgG such as IgG1, IgG2, IgG3, and IgG4, IgA, IgD, IgE, or IgM, but in a case of being used as a medicine, IgG and IgM are preferable.

Human antibodies include all antibodies having one or a plurality of variable and constant domains introduced from human immunoglobulin sequences. In one embodiment, all of the variable and constant domains are introduced from human immunoglobulin sequences (complete human antibodies).

The humanized antibody has a sequence different from a sequence of an antibody introduced from a non-human species by substitution, deletion, and/or addition of one or a plurality of amino acids, so that there is a low possibility for the humanized antibody to induce an immune response, and/or so that induction of a severe immune response is reduced, compared to the non-human species antibody, in a case of being administered to a human subject. In one embodiment, specific amino acids in a framework and constant domains of heavy chains and/or light chains of a non-human species antibody are mutated to produce a humanized antibody. In another embodiment, the constant domains of human antibodies are fused to variable domains of a non-human antibodies.

The chimeric antibody is an antibody in which variable domains and constant domains having different origins from each other are linked. For example, an antibody consisting of variable domains of heavy chains and light chains of a mouse antibody and the constant domains of heavy chains and light chains of a human antibody is a mouse/human heterologous chimeric antibody. It is possible to prepare a recombinant vector expressing a chimeric antibody by linking a DNA encoding the variable domains of a mouse antibody and a DNA encoding the constant domains of a human antibody, and incorporating thereof into an expression vector. It is possible to obtain a chimeric antibody produced during the culture by culturing a recombinant cell transformed with the vector and expressing the incorporated DNA.

A bispecific antibody is an antibody that recognizes two different antigenic specificities and are prepared by a chemical method or cell fusion. As a method for preparing a bispecific antibody, a method of preparing a bispecific antibody by binding two immunoglobulin molecules by using a cross-linking agent such as N-succinimidyl 3-(2-pyridyldithiol) propionate or S-acetylmercaptosuccinic acid anhydride, a method of preparing a bispecific antibody by binding Fab fragments of immunoglobulin molecules with each other, and the like have been reported.

An Fc fusion protein indicates a protein having an Fc domain and includes an antibody.

Fab is a monovalent fragment having V_{L}, V_{H}, C_{L} and C_{H}1 domains.

F(ab′)₂ is a bivalent fragment having two Fab fragments bound by a disulfide cross-linking at a hinge domain.

The Fv fragment has a single arm V_{L} and V_{H} domains of an antibody.

Single-chain antibody (scFv) is an antibody in which the V_{L} and V_{H} domains are joined via a linker (for example, a synthetic sequence of an amino acid residue) to form a continuous protein chain, in which the linker is long enough to fold the protein chain on itself and to form a monovalent antigen binding site.

A gene encoding a target protein can be obtained by a method known to those skilled in the art. In a case where the target protein is an antibody, it is possible to use a DNA encoding the L chain and a DNA encoding the H chain of the antibody.

It is possible to prepare the DNA encoding the L chain and the DNA encoding the H chain of the antibody as follows. An mRNA is extracted from a hybridoma, a cell, a phage, a ribosome, or the like having a gene that expresses an antibody. A cDNA is prepared by a reverse transcription reaction using a reverse transcriptase from the mRNA. The L chain gene or the H chain gene is amplified by PCR using a primer and a cDNA having a base sequence complementary to the L chain gene or the H chain gene, and each gene is obtained by binding to a cloning plasmid.

It is possible to prepare the DNA encoding the L chain fragment and the DNA encoding the H chain fragment of the antibody as follows. An mRNA is extracted from a hybridoma, a cell, a phage, a ribosome, or the like having a gene that expresses an antibody. A cDNA is prepared by a reverse transcription reaction using a reverse transcriptase from the mRNA. The L chain gene fragment or the H chain gene fragment is amplified by PCR using a primer having a base sequence complementary to the L chain gene fragment or the H chain gene fragment and a cDNA, and each gene fragment is obtained by binding to a cloning plasmid.

<SNAT2 and SNAT2 Genes>

In an aspect of the present invention, the origin of SNAT2 is not particularly limited, and a foreign gene encoding SNAT2 derived from a mammal such as human, monkey, mouse, rat, or hamster can be used. In an aspect of the present invention, the foreign gene refers to a gene introduced into an animal cell from outside. In addition, even in a case where an endogenous gene is amplified to introduce a gene, it is regarded as a foreign gene.

The base sequence and the amino acid sequence of human SNAT2 are shown in SEQ ID NOs: 1 and 2 in the sequence list.

Base sequence of human SNAT2 gene
(SEQ ID NO: 1)
ATGAAGAAGGCCGAAATGGGACGATTCAGTATTTCCCCGGATGAAGACAGC
AGCAGCTACAGTTCCAACAGCGACTTCAACTACTCCTACCCCACCAAGCAA
GCTGCTCTGAAAAGCCATTATGCAGATGTAGATCCTGAAAACCAGAACTTT
TTACTTGAATCGAATTTGGGGAAGAAGAAGTATGAAACAGAATTTCATCCA
GGTACTACTTCCTTTGGAATGTCAGTATTTAATCTGAGCAATGCGATTGTG
GGCAGTGGAATCCTTGGGCTTTCTTATGCCATGGCTAATACTGGAATTGCT
CTTTTTATAATTCTCTTGACATTTGTGTCAATATTTTCCCTGTATTCTGTT
CATCTCCTTTTGAAGACTGCCAATGAAGGAGGGTCTTTATTATATGAACAA
TTGGGATATAAGGCATTTGGATTAGTTGGAAAGCTTGCAGCATCTGGATCA
ATTACAATGCAGAACATTGGAGCTATGTCAAGCTACCTCTTCATAGTGAAA
TATGAGTTGCCTTTGGTGATCCAGGCATTAACGAACATTGAAGATAAAACT
GGATTGTGGTATCTGAACGGGAACTATTTGGTTCTGTTGGTGTCATTGGTG
GTCATTCTTCCTTTGTCGCTGTTTAGAAATTTAGGATATTTGGGATATACC
AGTGGCCTTTCCTTGTTGTGTATGGTGTTCTTTCTGATTGTGGTCATTTGC
AAGAAATTTCAGGTTCCGTGTCCTGTGGAAGCTGCTTTGATAATTAACGAA
ACAATAAACACCACCTTAACACAGCCAACAGCTCTTGTACCTGCTTTGTCA
CATAACGTGACTGAAAATGACTCTTGCAGACCTCACTATTTTATTTTCAAC
TCACAGACTGTCTATGCTGTGCCAATTCTGATCTTTTCATTTGTCTGTCAT
CCTGCTGTTCTTCCCATCTATGAAGAACTGAAAGACCGCAGCCGTAGAAGA
ATGATGAATGTGTCCAAGATTTCATTTTTTGCTATGTTTCTCATGTATCTG
CTTGCCGCCCTCTTTGGATACCTAACATTTTACGAACATGTTGAGTCAGAA
TTGCTTCATACCTACTCTTCTATCTTGGGAACTGATATTCTTCTTCTCATT
GTCCGTCTGGCTGTGTTAATGGCTGTGACCCTGACAGTACCAGTAGTTATT
TTCCCAATCCGGAGTTCTGTAACTCACTTGTTGTGTGCATCAAAAGATTTC
AGTTGGTGGCGTCATAGTCTCATTACAGTGTCTATCTTGGCATTTACCAAT
TTACTTGTCATCTTTGTCCCAACTATTAGGGATATCTTTGGTTTTATTGGT
GCATCTGCAGCTTCTATGTTGATTTTTATTCTTCCTTCTGCCTTCTATATC
AAGTTGGTGAAGAAAGAACCTATGAAATCTGTACAAAAGATTGGGGCTTTG
TTCTTCCTGTTAAGTGGTGTACTGGTGATGACCGGAAGCATGGCCTTGATT
GTTTTGGATTGGGTACACAATGCACCTGGAGGTGGCCAT
Amino acid sequence of human SNAT2 gene
(SEQ ID NO: 2)
MKKAEMGRFSISPDEDSSSYSSNSDFNYSYPTKQAALKSHYADVDPENQNF
LLESNLGKKKYETEFHPGTTSFGMSVFNLSNAIVGSGILGLSYAMANTGIA
LFIILLTFVSIFSLYSVHLLLKTANEGGSLLYEQLGYKAFGLVGKLAASGS
ITMQNIGAMSSYLFIVKYELPLVIQALTNIEDKTGLWYLNGNYLVLLVSLV
VILPLSLFRNLGYLGYTSGLSLLCMVFFLIVVICKKFQVPCPVEAALIINE
TINTTLTQPTALVPALSHNVTENDSCRPHYFIFNSQTVYAVPILIFSFVCH
PAVLPIYEELKDRSRRRMMNVSKISFFAMFLMYLLAALFGYLTFYEHVESE
LLHTYSSILGTDILLLIVRLAVLMAVTLTVPVVIFPIRSSVTHLLCASKDF
SWWRHSLITVSILAFTNLLVIFVPTIRDIFGFIGASAASMLIFILPSAFYI
KLVKKEPMKSVQKIGALFFLLSGVLVMTGSMALIVLDWVHNAPGGGH

As a foreign gene encoding SNAT2,
(1) a gene encoding a protein consisting of an amino acid sequence of SEQ ID NO: 2;
(2) a gene encoding a protein consisting of an amino acid sequence in which one or several amino acids are deleted, substituted, or added in the amino acid sequence of SEQ ID NO: 2, and having SNAT2 function; or
(3) a gene encoding a protein consisting of an amino acid sequence having 85% or more (more preferably 90% or more, particularly preferably 95% or more, most preferably 98% or more) sequence identity with the amino acid sequence of SEQ ID NO: 2, and having SNAT2 function;

can be used.

The SNAT2 function means a function of activating the mTORC1/S6K pathway. In rat cells, it is known that activation of mTOR suppresses cell proliferation and increases cell size.

As a foreign gene encoding SNAT2,
(4) a gene consisting of a base sequence of SEQ ID NO: 1;
(5) a gene consisting of a base sequence in which one or several bases are deleted, substituted or added in the base sequence of SEQ ID NO: 1, and encoding a protein having SNAT2 function;
(6) a gene consisting of a base sequence that hybridizes under stringent conditions to a sequence complementary to the base sequence of SEQ ID NO: 1 and encoding a protein having SNAT2 function; or
(7) a gene having a base sequence having 90% or more (more preferably 95% or more, particularly preferably 98% or more) sequence identity with the base sequence of SEQ ID NO: 1 and encoding a protein having SNAT2 function:
can also be used.

“One or several” in “amino acid sequence in which one or several amino acids are deleted, substituted or added” means preferably 1 to 20, more preferably 1 to 10, further more preferably 1 to 5, and particularly preferably 1 to 3.

The sequence identity in an aspect of the present invention indicates a value calculated by the following formula.

% Sequence identity=[(number of identical residues)/(longer alignment length)]×100

The sequence identity between two amino acid sequences can be determined by any method known to those skilled in the art, and can be determined using the Basic Local Alignment Search Tool (BLAST) program (J. Mol. Biol. 215: 403-410, 1990) or the like. The “longer alignment length” of the denominator means that in a case where two alignments are compared, the longer alignment length is used as the denominator.

“One or several” in the “base sequence in which one or several bases are deleted, substituted or added” means preferably 1 to 20, more preferably 1 to 10, further more preferably 1 to 5, and particularly preferably 1 to 3.

“Under the stringent conditions” in “hybridizes under stringent conditions” means hybridizing under moderately or highly stringent conditions, which can be recognized by those skilled in the art. Examples of the moderately stringent conditions include conditions described in Sambrook et al., Molecular Cloning: A Laboratory Manual, Third Edition, Vol. 1, 7. 42-7. 45 Cold Spring Harbor Laboratory Press, 2001. Examples of the moderately stringent conditions include a hybridization conditions of a pre-wash solution of 5×SSC, 0.5% SDS, and 1.0 mmol/L EDTA (pH 8.0), approximately 50% formamide at approximately 40° C. to 50° C., and 2×SSC to 6×SSC (or other similar hybridization solutions such as Stark's solution in approximately 50% formamide at approximately 42° C.) in a nitrocellulose filter, and washing conditions of approximately 60° C., 0.5×SSC, and 0.1% SDS. Highly stringent conditions can also be easily determined by those skilled in the art, and examples thereof include hybridization and/or washing at a higher temperature and/or a lower salt concentration than the above-described moderately stringent conditions. For example, the above-described hybridization conditions and washing at 68° C., 0.2×SSC, and 0.1% SDS can be exemplified. Here, the composition of 1×SSC is 150 mmol/L NaCl, 15 mmol/L sodium citrate, and pH 7.4. SDS is sodium dodecyl sulfate, and EDTA is ethylene diamine tetraacetic acid.

Information on the amino acid sequence and the base sequence of non-human SNAT2 is shown below. The following numbers indicate the Gene No. of National Center for Biotechnology Information (NCBI).

ID: 29642 rat (Rattus norvegicus)

ID: 67760 mouse (Mus musculus)

ID: 566537 zebrafish (Danio rerio)

ID: 338044 cattle (Bos taurus)

ID: 417807 chicken (Gallus gallus)

ID: 702253 Rhesus monkey (Macaca mulatta)

ID: 101083348 domestic cat (Felis catus)

ID: 100525644 pig (Sus scrofa)

ID: 101149564 gorilla (Gorilla gorilla)

For non-human SNAT2, as in the case of human SNAT2,
(2A) a gene encoding a protein consisting of an amino acid sequence in which one or several amino acids are deleted, substituted or added in a predetermined amino acid sequence, and having SNAT2 function;
(3A) a gene encoding a protein consisting of an amino acid sequence having 85% or more (more preferably 90% or more, particularly preferably 95% or more, and most preferably 98% or more) sequence identity with a predetermined amino acid sequence, and having SNAT2 function;
(5A) a gene encoding a protein consisting of a base sequence in which one or several bases are deleted, substituted or added in a predetermined base sequence and having SNAT2 function;
(6A) a gene encoding a protein consisting of a base sequence that hybridizes under stringent conditions to a sequence complementary to a predetermined base sequence, and having SNAT2 function; or
(7A) a gene having a base sequence having 90% or more (more preferably 95% or more, particularly preferably 98% or more) sequence identity with a predetermined base sequence and encoding a protein having SNAT2 function:
may be used.

The foreign gene encoding SNAT2 is linked to the promoter.

The promoter is not particularly limited as long as it can function in animal cells of a host and make the animal cells to express SNAT2. The promoter is preferably cytomegalovirus promoter (CMV promoter), EF1α promoter (promoter of human polypeptide chain elongation factor gene), Simian Virus 40 promoter (SV40 promoter), β-actin promoter, MMLV-LTR promoter (promoter of long terminal repeat of moloney murine leukemia virus), or mouse β-globin promoter, and the CMV promoter is more preferable.

In the animal cells of an aspect of the present invention, SNAT2 is overexpressed. Overexpression means that expression of a certain gene exceeds a normal expression level in a host. By introducing a foreign gene encoding SNAT2 into a host and making the foreign gene express SNAT2 in the host, an animal cell overexpressing SNAT2 can be obtained.

The expression level of the SNAT2 in the animal cells of an aspect of the present invention is preferably 3 times or more, more preferably 3.5 times or more, further more preferably 4 times or more, more preferably 4.5 times or more, further more preferably 5 times or more, and particularly preferably 5.5 times or more that of animal cells not having a foreign gene encoding SNAT2. There may be no upper limit, but it may be 30,000 times or less, or may be 10,000 times or less.

The expression level of the SNAT2 can be examined by reverse transcription-polymerase chain reaction (RT-PCR) method and the like. Measurement of the expression level of SNAT2 is preferably carried out by reverse transcription of mRNA and real-time PCR. The expression level of the SNAT2 is preferably a relative expression level calculated by normalization. Normalization can be performed, for example, by comparative quantification using the expression level of a housekeeping gene such as β-actin or HPRT1 as an endogenous control.

<Animal Cells>

The cells in an aspect of the present invention are not particularly limited as long as the cells are animal cells. Examples of the animal cells include Chinese hamster ovary (CHO) cells, BHK cells, 293 cells, myeloma cells (such as NS0 cells), PerC6 cells, SP2/0 cells, hybridoma cells, COS cells, 3T3 cells, HeLa cells, Vero cells, MDCK cells, PC12 cells, and WI38 cells. Among these, particularly CHO cells, BHK cells, 293 cells, myeloma cells (such as NS0 cells), PerC6 cells, SP2/0 cells, and hybridoma cells are preferable, and CHO cells are more preferable. The CHO cells are widely used for production of recombinant proteins such as cytokines, coagulation factors, and antibodies. It is preferable to use CHO cells deficient in dihydrofolate reductase (DHFR), and as DHFR-deficient CHO cells, for example, CHO-DG44 can be used.

[Method for Producing Animal Cells]

According to an aspect of the present invention, there is provided a method for producing an animal cell of an aspect of the present invention, including a step of introducing a gene encoding a target protein and a foreign gene encoding SNAT2 and linked to a promoter into animal cells.

It is preferable that the gene encoding a target protein and the foreign gene encoding SNAT2 are each incorporated into a vector and introduced into a host animal cell.

As the vector that can be used to introduce a gene into a host, it is possible to use a mammalian-derived expression vector. Examples thereof include pCMV6-Entry (manufactured by OriGene Technologies, Inc.), pcDNA3 (manufactured by Invitrogen), pEGF-BOS (Nucleic Acids. Res. 1990, 18 (17), p 5322), pEF, pCDM8 (manufactured by Funakoshi Co., Ltd.), INPEP4 (manufactured by Biogen-IDEC), and the like, but are not particularly limited thereto.

In addition, it is known that mRNA having polyA is stable in cells. The gene encoding a target protein and the foreign gene encoding SNAT2 may have polyA signals necessary for adding polyA to the gene, for example, mouse β-globin polyA signal, bovine growth hormone polyA signal, SV40 polyA signal, and the like.

The method for introducing a gene encoding a target protein and a foreign gene encoding SNAT2 into animal cells is not particularly limited, and the introduction can be performed by a method known to those skilled in the art. For example, it is possible to perform electroporation, lipofection, a calcium phosphate method, a DEAE dextran method, a method using cationic liposome DOTAP (manufactured by Roche diagnostics), or a method using a virus vector. Among these, electroporation is preferable.

In a case where a gene is introduced into an animal cell, the gene is introduced into only some of the cells subjected to gene introduction, depending on the kind of the expression vector used and the gene introduction method. In order to identify and select cells into which the gene has been introduced, for example, a gene encoding a selectable marker for resistance to antibiotics may be introduced into the host cell along with the target gene. Preferable selectable markers include those conferring resistance to drugs, such as G418, hygromycin, and methotrexate.

In the cells of an aspect of the present invention, the gene encoding a target protein may be expressed in a transient expression system, or may be expressed in a constant expression system, but may be preferably expressed in the constant expression system.

In the cells of an aspect of the present invention, the foreign gene encoding SNAT2 may be expressed in a transient expression system, or may be expressed in a constant expression system, but may be preferably expressed in the constant expression system.

The transient expression system is a system in which a cyclic plasmid is incorporated into cells and made to express by a calcium phosphate method, an electroporation method, a lipofection method, or the like. A gene encoding a target protein and a foreign gene encoding SNAT2 often exist outside the chromosome.

The constant expression system is a system in which a cyclic plasmid or a linear plasmid created by restriction enzyme treatment is incorporated into cells by a calcium phosphate method, an electroporation method, a lipofection method, or the like, and a part thereof is inserted into a cell genome to express a target protein. It is possible to maintain expression of the gene encoding a target protein and the foreign gene encoding SNAT2 for a long period of time. In addition, drug selection is possible by introduction of a drug resistant gene into a plasmid, and it is possible to efficiently select the cells in which the gene encoding a target protein and the foreign gene encoding SNAT2 are maintained on chromosomes.

[Method for Producing Target Protein]

According to the present invention, there is provided a method for producing a target protein, including culturing an animal cell of an aspect of the present invention.

It is possible to produce a target protein by culturing the animal cell of an aspect of the present invention. A culture can be performed according to a known method.

As a medium used for culturing animal cells of an aspect of the present invention, a medium used for culturing normal animal cells can be used. For example, an OptiCHO (Life Technologies, 12681011) medium, a Dulbecco's modified eagle medium (DMEM), an eagle's minimum essential medium (MEM), an RPMI-1640 medium, an RPMI-1641 medium, a F-12K medium, a Ham F12 medium, an Iscove's modified method Dulbecco's medium (IMDM), a McCoy's 5A medium, a Leibovitz L-15 medium, and an EX-CELL (trademark) 300 series (JRH Biosciences), a CHO-S-SFMII (Invitrogen Corporation), a CHO-SF (Sigma-Aldrich), a CD-CHO (Invitrogen Corporation), an IS CHO-V (Irvine Scientific), a PF-ACF-CHO (Sigma-Aldrich Corporation), or the like can be used.

Serum such as fetal calf serum (FCS) may be added to the medium, and culture in a serum-free medium may be more preferable and culture in a total synthesis medium may be most preferable.

The medium may be supplemented with additional components such as amino acids, salts, sugars, vitamins, hormones, proliferation factors, buffers, antibiotics, lipids, trace elements, and hydrolysates of plant proteins.

The pH of the medium varies depending on the cells to be cultured, but is generally pH 6.0 to 8.0, preferably pH 6.8 to 7.6, and more preferably pH 7.0 to 7.4.

The culture temperature is generally 30° C. to 40° C., preferably 32° C. to 37° C., and more preferably 36° C. to 37° C., and the culture temperature may be changed during the culture.

The culture is preferably performed in an atmosphere having a CO₂ concentration of 0% to 40%, and preferably 2% to 10%.

The culture time is not particularly limited, but is generally 12 hours to 90 days, preferably 24 hours to 60 days, more preferably 24 hours to 30 days.

In the culture, the medium can be replaced, aerated, and agitated depending on the necessity.

The animal cells of an aspect of the present invention can be cultured in a culture device (also referred to as a bioreactor) or other suitable containers. Examples of the culture device include a fermenter tank culture device, an air lift culture device, a culture flask culture device, a spinner flask culture device, a microcarrier culture device, a fluidized bed culture device, a hollow fiber culture device, a roller bottle culture device, a filling tank culture device, and the like.

The culture scale is generally 1 L to 20,000 L, preferably 1 L to 10,000 L, more preferably 200 L to 2,000 L, and further more preferably 500 L to 2,000 L.

As the culture, any method such as batch culture, fed-batch culture (also referred to as feeding culture), or perfusion culture may be used, but fed-batch culture or perfusion culture is preferable.

The batch culture is a discontinuous method in which cells are proliferated for a short period in a fixed-volume culture medium, and then are completely harvested. A culture product proliferated using the batch method experiences an increase in cell density until reaching a maximum cell density, and then medium components are consumed and levels of metabolic by-products (lactate, ammonia, and the like) accumulate, which causes a viable cell density to decline. Harvesting is typically performed at a time when the maximum cell density (typically, 5 to 10×10⁶ cells/mL) is achieved. A batch process is the simplest culture method, but the viable cell density is limited by nutrient availability, and once the cells reach the maximum density, the culture is declined and the production of the target protein is reduced. Since accumulation of waste products and nutrient depletion rapidly lead to culture decline (typically approximately 3 to 7 days), it is not possible to extend the duration of production of the target protein.

The fed-batch culture is a culture method that improves the batch process by feeding a bolus or continuous medium and replenishing the consumed medium components. That is, in the fed-batch culture, the culture form is suspension culture, which provides additional components to the culture at one or more points after the start of the culture process. Examples of the additional components include nutrition supplement components for cells that are depleted during the culture process, and may include other auxiliary components (for example, cell cycle inhibiting compounds).

In the fed-batch culture, since additional nutrients are added throughout the culture period, there is a possibility that higher cell densities and higher yields of the target protein may be achieved compared to the batch culture. In the fed-batch culture, unlike the batch culture, it is possible to prepare and maintain a two-phase culture by operating the feeding schedule and the culture components such that a period (proliferation phase) of cell proliferation for achieving a desired cell density is distinguished from a period (production period) of stopped or slow cell proliferation. In this manner, there is a possibility that the fed-batch culture can achieve a higher production amount of the target protein compared to the batch culture.

In fed-batch culture, a seeded cell density of the cell culture is generally 0.2×10⁶ cells/mL to 1×10⁷ cells/mL, preferably 0.2×10⁶ cells/mL to 5×10⁶ cells/mL, more preferably 0.5×10⁶ cells/mL to 2.5×10⁶ cells/mL, and further more preferably 0.5×10⁶ cells/mL to 1.5×10⁶ cells/mL.

In the fed-batch culture, the viable cell rate during the culture period is preferably 60% to 100%, more preferably 70% to 100%, and further more preferably 75% to 100% in the entire period.

A perfusion culture is a culture method in which a fresh medium is added and a used medium is removed at the same time, and there is a possibility that the batch culture and the fed-batch culture can be further improved. The perfusion culture can be performed using an Alternating Tangential Flow Filtration (ATF) pump or a Tangential Flow Filtration (TFF) pump, for example. According to the perfusion culture, it is possible to achieve a high cell density exceeding 1×10⁸ cells/mL. A typical perfusion culture starts with a batch culture start-up lasting one or two days, then, a fresh feed medium is continuously, in phases, and/or intermittently added to the culture product, and the used medium is removed at the same time. In the perfusion culture, it is possible to remove a used medium while maintaining a cell density using a method such as sedimentation, centrifugation, or filtration. The perfusion culture can be performed using an ATF pump or a TFF pump, for example.

The advantage of the perfusion culture is that the culture in which a target protein is produced is maintained for a longer period than the batch culture method or the fed-batch culture. However, in order to maintain a long-term perfusion culture, particularly a perfusion culture at a high cell density, preparation, use, storage, and disposal of a medium are required. In the perfusion culture, many nutrients are required, and the production cost of the target protein tends to be higher than that in the batch culture and the fed-batch culture. In addition, since it is possible to continue culture while harvesting antibodies out of the system by selecting a membrane pore diameter, it is possible to keep the quality of the antibody high by shortening a retention time for the antibody and reducing chemical change of the antibody, in a culture solution.

It is also possible to perform culture in which the fed-batch culture and the perfusion culture are combined to each other. As an example, it is possible to use fed-batch culture with a bolus feed to maintain the culture of cells in the proliferation period, and subsequently, it is possible to use perfusion culture to produce a target protein.

Perfusion may be in any form of continuous perfusion, phased perfusion, intermittent perfusion, or a combination thereof. Animal cells are retained in the culture product and the used medium that is removed may substantially do not include cells or may have much fewer cells than the culture product. The target protein expressed by a cell culture can be retained in the culture product or harvested by selecting the membrane pore diameter. To prevent the cell density during the culture from becoming excessive, a part of the culture solution may be extracted together with the cells, and the cell density may be reduced (cell bleeding) by adding the same amount of the fresh medium.

In the perfusion culture, the seeded cell density of the cell culture is generally 0.2×10⁶ cells/mL to 3×10⁷ cells/mL, preferably 0.2×10⁶ cells/mL to 1×10⁷ cells/mL, and more preferably 0.5×10⁶ cells/mL to 1×10⁷ cells/mL.

In the perfusion culture, the viable cell rate during the culture period is preferably 80% or more, more preferably 85% or more, and further more preferably 90% or more in the entire period.

In the perfusion culture, the highest attainable cell density is preferably 2×10⁸ cells/mL or less, more preferably 1.5×10⁸ cells/mL or less, and further more preferably 1.0×10⁸ cells/mL or less.

The perfusion ratio in the perfusion culture is preferably 0.3 vvd to 5.0 vvd, and more preferably 0.3 vvd to 1.5 vvd. Here, vvd represents the following.

vvd=(volume of fresh medium/working volume of reactor/day, feed amount of culture solution/amount of culture solution per day)

The seeded cell density in the cell culture and the highest attainable cell density in the culture can be obtained by measuring the number of cells by a conventional method and dividing the number of cells by the amount of the culture solution.

The viable cell rate (survival rate) during the culture period is obtained by dividing the number of viable cells by (the number of viable cells+the number of dead cells). For example, the number of cells can be measured using Vi-CELL XR (Beckman Coulter, Inc.).

The target protein produced by the above-described culture can be purified. The separation and purification of the target protein may be performed by using separation and purification methods used for normal proteins. For example, it is possible to perform separation and purification of the target protein by appropriately selecting and combining a chromatography column such as affinity chromatography, a filter, ultrafiltration, salting out, dialysis, sodium dodecyl sulfate (SDS) polyacrylamide gel electrophoresis, isoelectric focusing, or the like, but is not limited thereto. The concentration of the target protein obtained as described above can be measured by absorbance measurement or enzyme-linked immunosorbent assay (ELISA) or the like.

Columns used for affinity chromatography include a protein A column, a protein G column, and the like. Examples of the chromatography other than affinity chromatography include ion exchange chromatography, hydrophobic chromatography, gel filtration, reverse phase chromatography, adsorption chromatography, and the like. The chromatography can be performed using liquid phase chromatography such as high performance liquid chromatography (HPLC) or fast protein liquid chromatography (FPLC).

In addition, it is possible to modify the target protein or partially remove the peptide by causing appropriate polypeptide modifying enzyme to act on before or after purification. As the polypeptide modifying enzyme, trypsin, chymotrypsin, lysyl endopeptidase, protein kinase, glucosidase, and the like are used, for example.

[Use of Target Protein]

In a case where the target protein produced by the method of an aspect of the present invention has useful biological activity as a pharmaceutical, it is possible to produce pharmaceuticals by mixing the target protein with a pharmaceutically acceptable carrier or additive and formulating thereof.

Examples of pharmaceutically acceptable carriers and additives include water, pharmaceutically acceptable organic solvents, collagen, polyvinyl alcohol, polyvinyl pyrrolidone, carboxy vinyl polymer, sodium carboxymethylcellulose, sodium polyacrylate, sodium alginate, aqueous dextran, sodium carboxymethyl starch, pectin, methylcellulose, ethylcellulose, xanthan gum, gum arabic, casein, agar, polyethylene glycol, diglycerin, glycerin, propylene glycol, petrolatum, paraffin, stearyl alcohol, stearic acid, human serum albumin (HSA), mannitol, sorbitol, lactose, pharmaceutically acceptable surfactants, and the like.

For example, in a case of being used as an injectable preparation, it is possible to dissolve the purified target protein in a solvent such as physiological saline, a buffer solution, a glucose solution, or the like, and to use those obtained by adding an anti-adsorption agent such as Tween80, Tween20, gelatin, human serum albumin, or the like thereto. Alternatively, the target protein may be a freeze-dried product used for dissolving and reconstituting the product before use, and sugar alcohol or sugars such as mannitol or glucose can be used as an excipient for freeze-drying, for example.

A method of administering the target protein may be either of oral administration or parenteral administration, but is preferably parenteral administration. Examples thereof include injection (for example, systemic or local administration by intravenous injection, intramuscular injection, intraperitoneal injection, subcutaneous injection, and the like), nasal administration, pulmonary administration, transdermal administration, or the like.

A dose of the target protein is appropriately selected depending on the kind of the target protein, the kind of the disease to be treated or prevented, the age of the patient, the severity of the disease, and the like. Generally, the dose is in a range of 0.001 mg to 1,000 mg per kg of body weight at a time, but is not particularly limited thereto.

The present invention will be described more specifically with reference to the following examples, but the present invention is not limited to the examples.

### EXAMPLES

### <Example 1> Preparation of animal cell

A vector encoding a human SNAT2 gene (OriGene Technologies, Inc., Cat #RC201892) was purchased, and the SNAT2 gene expression cassette (promoter, open reading frame, terminator) was amplified by polymerase chain reaction (PCR) using primer 1 (SEQ ID NO: 3) and primer 2 (SEQ ID NO: 4).

The amplified SNAT2 expression cassette was inserted into a vector co-expressing L chain and H chain of each antibody of antibody 1 (denosumab, https: //www.drugbank.ca/drugs/DB06643), antibody 2 (blosozumab, https://www.drugbank.ca/drugs/DB12560), or antibody 3 (anti-MUC1 antibody, JP 2016-517691A). Construction of the L chain and H chain co-expression vector and introduction thereof into the cell were performed according to Example 2 of JP2016-517691A.

All host CHO-DG44 cells prior to the introduction of genes of the antibody and SNAT2 were cultured in an incubator at 37° C. in a 5% CO₂ atmosphere. The vector was introduced into host cells 5×10⁶ cells using an electroporation method (Lonza Group AG, 4D-Nucleofector), suspended in 20 mL of an OptiCHO (Life Technologies, 12681011) medium containing 100 times-diluted HT Supplement (100×) (Life Technologies, 11067-030), and then seeded in a T75 flask. After one day, the medium was replaced with an OptiCHO (Life technologies, 12681011) medium containing 175 nmol/L methotrexate (MTX) (Wako Pure Chemical Corporation) without HT supplements. Microscopic observation of the T75 flask was started 3 weeks after the gene introduction, the culture volume was sequentially expanded to 20 mL from the flask in which cell proliferation was observed, seeded in a 125 mL shaking flask (Corning), and cultured with shaking at 140 rpm.

By the above method, antibody 1-SNAT2 cells, antibody 2-SNAT2 cells, and antibody 3-SNAT2 cells that forcibly express the human SNAT2 gene, and the antibody 1, antibody 2, or antibody 3 were established. In addition, gene introduction was carried out in the same manner using a vector expressing antibody L chain and H chain to construct antibody 1 cell, antibody 2 cell, and antibody 3 cell in the control group.

Primer-1:
(SEQ ID NO: 3)
CCGCGGTCATAGCTGTTTCCTGAAC
Primer-2:
(SEQ ID NO: 4)
CAGCTATGACCGCGGTTAATGGCCACCTCCAGGTGCATTGTGT

### <Example 2> Culture Test

Feeding culture test was carried out using 8 levels of antibody 1-SNAT2 cells, antibody 2-SNAT2 cells, antibody 3-SNAT2 cells and antibody 1 cell, antibody 2 cells, and antibody 3 cells.

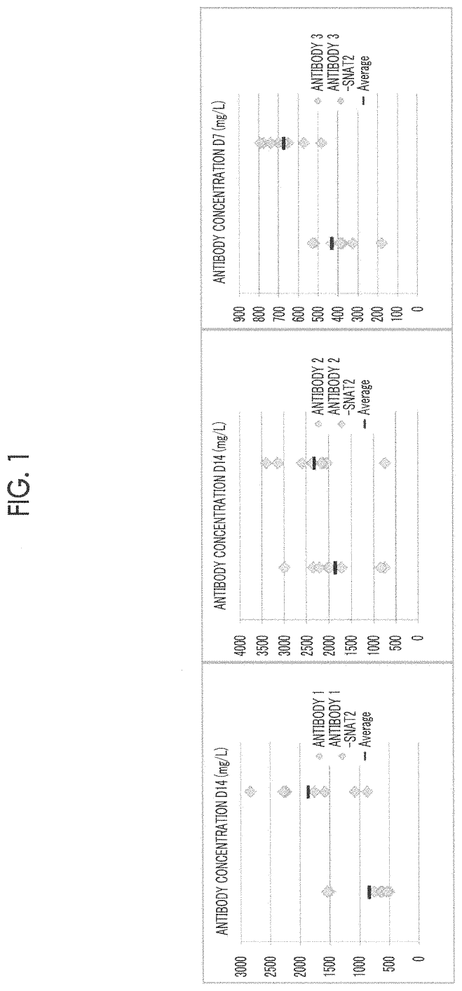

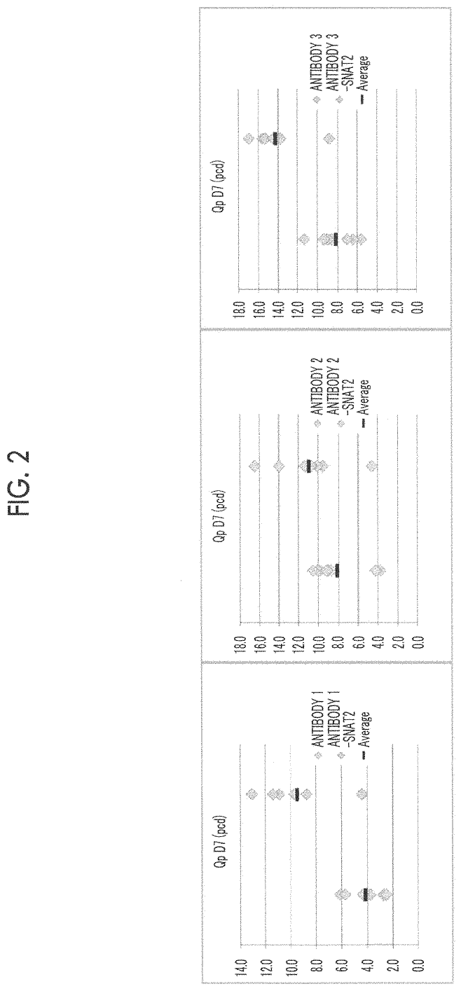

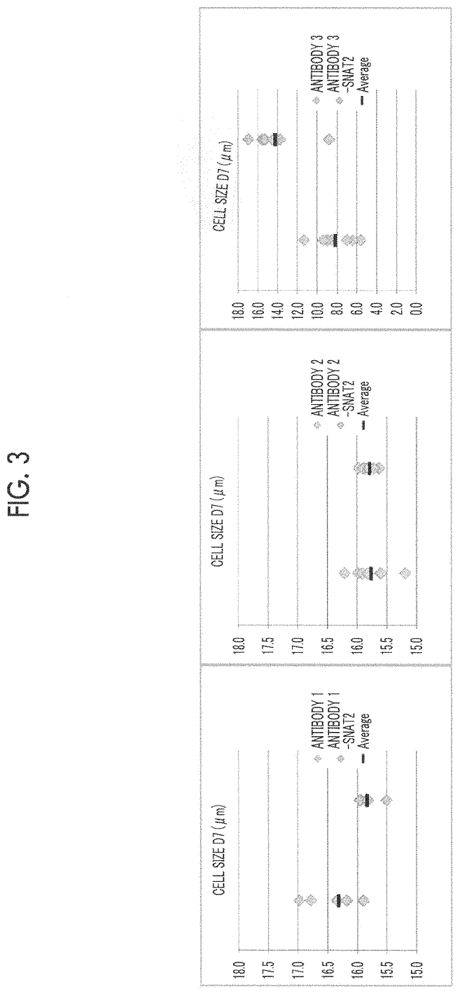

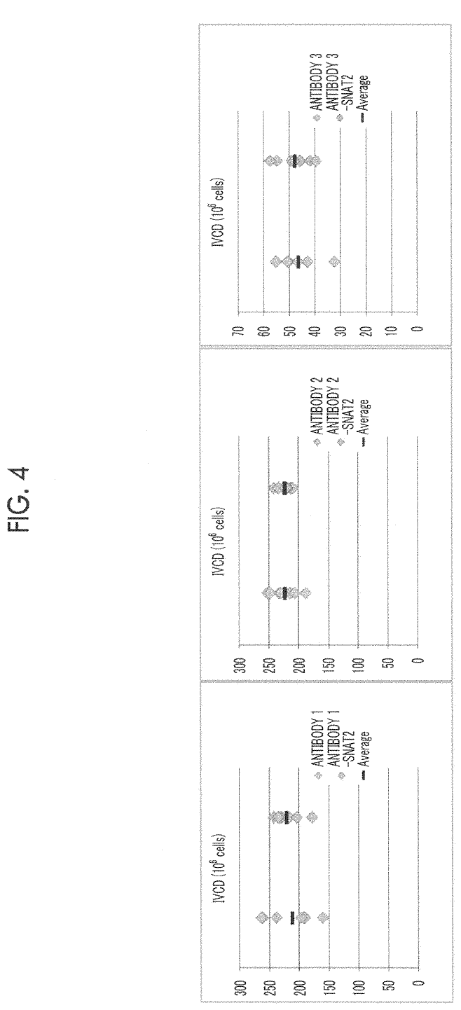

The cells were suspended in 15 mL of an OptiCHO (Life Technologies, 12681011) medium at a cell density of 5×10⁵ cells/mL and automatically cultured with an AMBR15 culture device (Sartorius AG). From the second day to the 13th day after the start of culture, 2% of a feed medium (Cellboost 7a, 7b, GE healthcare) at initial culture volume ratio was added every day. Sampling was performed every third to fourth days to measure the cell density, culture solution components, and an antibody concentration over time. On the 14th day from the start of the culture, the culture solution was harvested, and cells and cell debris were removed using a 0.22 μm-depth filter (Merck Millipore Corporation). In a case where the antibody concentration in the supernatant and the antibody productivity (Qp) per cell were measured by liquid chromatography, the antibody concentration increased by 121% for antibody 1, 25% for antibody 2, and 56% for antibody 3 (FIG. 1 and Table 1). In addition, Qp increased by 127% for antibody 1, 34% for antibody 2, and 74% for antibody 3 (FIG. 2 and Table 2). It is considered that the antibody productivity was improved since the protein biosynthesis was activated by the expression of SNAT2. On the other hand, as a result of measuring the cell diameter with Vi-CELL (Beckman Coulter, Inc.), surprisingly, it was found that the size of each cell was antibody 1-SNAT2 cells (average 15.8 μm), antibody 2-SNAT2 cells (average 16.3 μm), and antibody 3-SNAT2 cells (average 16.5 μm), and compared to antibody 1 cells (average 16.3 μm), antibody 2 cells (average 15.8 μm), and antibody 3 cells (average 16.2 μm) of the control group, the cell diameter did not necessarily increase and showed almost the same level (FIG. 3 and Table 3). In addition, the cumulative number of viable cells (IVCD) during the culture period showing cell proliferation property was similar to or slightly higher than that of the control group (FIG. 4 and Table 4). In FIGS. 1 to 4, only the antibody is shown on the left, and antibody-SNAT2 is shown on the right. This proved that the expression of SNAT2 did not enhance the differentiation of CHO cells, and the cells maintained the cell proliferation ability required for the establishment of antibody-producing cells and the production of antibodies.

The integral viable cell density (IVCD) was measured by the following method. The viable cell density (VCD) is measured by Vi-CELL XR (Beckman Coulter, Inc.) on days 0, 3, 7, 10 and 14, and the respective values are denoted as V0, V3, V7, V10, and V14. The itegrated viable cell density (IVCD) is calculated by the following formula.

$$Integral viable cell density ( IVCD ) = \{ ( V 0 + V 3 ) \times ( 3 \text{-} 0 ) / 2 \} + \{ ( V 3 + V 7 ) \times ( 7 \text{-} 3 ) / 2 \} + \{ ( V 10 + V 7 ) \times ( 10 \text{-} 7 ) / 2 \} + \{ ( V 14 + V 10 ) \times ( 14 \text{-} 10 ) / 2 \}$$

TABLE 1
Antibody concentration D14 comparison

| | |
|---|---|
| Antibody 1 | 100% |
| Antibody 1-SNAT2 | 221% |

Antibody concentration D14 comparison

| | |
|---|---|
| Antibody 2 | 100% |
| Antibody 2-SNAT2 | 125% |

Antibody concentration D7 comparison

| | |
|---|---|
| Antibody 3 | 100% |
| Antibody 3-SNAT2 | 156% |

TABLE 2
Qp D7 (pcd) comparison

| | |
|---|---|
| Antibody 1 | 100% |
| Antibody 1-SNAT2 | 227% |

Qp D7 (pcd) comparison

| | |
|---|---|
| Antibody 2 | 100% |
| Antibody 2-SNAT2 | 134% |

Qp D7 (pcd) comparison

| | |
|---|---|
| Antibody 3 | 100% |
| Antibody 3-SNAT2 | 174% |

TABLE 3
Cell size D7 comparison

| | |
|---|---|
| Antibody 1 | 100% |
| Antibody 1-SNAT2 | 97% |

Cell size D7 comparison

| | |
|---|---|
| Antibody 2 | 100% |
| Antibody 2-SNAT2 | 100% |

Cell size D7 comparison

| | |
|---|---|
| Antibody 3 | 100% |
| Antibody 3-SNAT2 | 102% |

TABLE 4
IVCD D14 comparison

| | |
|---|---|
| Antibody 1 | 100% |
| Antibody 1-SNAT2 | 105% |

IVCD D14 comparison

| | |
|---|---|
| Antibody 2 | 100% |
| Antibody 2-SNAT2 | 100% |

IVCD D7 comparison

| | |
|---|---|
| Antibody 3 | 100% |
| Antibody 3-SNAT2 | 103% |

The antibody productivity Qp [pg/cell/day] per cell was obtained by the following formula.

$${Q}_{p} = - \frac{{P}_{t 2} - {P}_{t 1}}{{\int }_{t 1}^{t 2} X dt}$$

Pti [g/L]=concentration of purified product on culture day ti

Xti [cell/day]=viable cell density on culture day ti

An approximate value of integration was calculated by obtaining the area under the proliferation curve from time t1 to t2 as the area of a trapezoid.

$${\int }_{t 1}^{t 2} X dt ≅ \sum \frac{( {t}_{2} - {t}_{1} ) ( {X}_{t 2} + {X}_{t 1} )}{2}$$

[Sequence list] The international patent application based on the International Patent Cooperation Treaty.

What is claimed is:

1. An animal cell comprising: a gene encoding a target protein; and
a foreign gene encoding SNAT2 and linked to a promoter, wherein the SNAT2 is overexpressed.

2. The animal cell according to claim 1,
wherein the overexpression is constant.

3. The animal cell according to claim 1,
wherein the foreign gene encoding SNAT2 has a base sequence having 90% or more sequence identity with a base sequence of SEQ ID NO: 1.

4. The animal cell according to claim 1,
wherein the foreign gene encoding SNAT2 has a base sequence of SEQ ID NO: 1.

5. The animal cell according to claim 1,
wherein the animal cell is a CHO cell.

6. A method for producing the animal cell according to claim 1, the method comprising:
a step of introducing a gene encoding a target protein and a foreign gene encoding SNAT2 and linked to a promoter, into animal cells.

7. The method according to claim 6,
wherein the step of introducing a foreign gene encoding SNAT2 and linked to a promoter is performed by electroporation.

8. A method for producing a target protein, comprising:
culturing the animal cell according to claim 1.

9. The method according to claim 8,
wherein the culture is fed-batch culture.

10. The method according to claim 9,
wherein a seeded cell density of the cell culture is 0.2×10⁶ cells/mL to 5×10⁶ cells/mL.

11. The method according to claim 10,
wherein a viable cell rate during a culture period is 60% or more in the entire period.

12. The method according to claim 8,
wherein the culture is perfusion culture.

13. The method according to claim 12,
wherein a seeded cell density of the cell culture is 0.2×10⁶ cells/mL to 1×10⁷ cells/mL.

14. The method according to claim 13,
wherein a viable cell rate during a culture period is 90% or more in the entire period.
